(19) United States
(12) Patent Application Publication
(10) Pub. No.: US 2016/0345804 A1
(43) Pub. Date: Dec. 1, 2016

(54) SWING PRISM, PRISM MOUNTING ARRANGEMENT AND ENDOSCOPE WITH VARIABLE VIEWING DIRECTION

(71) Applicant: OLYMPUS WINTER & IBE GMBH, Hamburg (DE)

(72) Inventors: Martin WIETERS, Barsbuettel (DE); Jens ROSE, Hamburg (DE)

(73) Assignee: OLYMPUS WINTER & IBE GMBH, Hamburg (DE)

(21) Appl. No.: 15/233,160

(22) Filed: Aug. 10, 2016

Related U.S. Application Data
(63) Continuation of application No. PCT/EP2015/052489, filed on Feb. 6, 2015.

(30) Foreign Application Priority Data
Feb. 13, 2014 (DE) 102014202612.1

(51) Int. Cl. A61B 1/00; G02B 23/24; G02B 26/08; A61B 1/04; G02B 23/02

(52) U.S. Cl. CPC A61B 1/00096; A61B 1/0011; A61B 1/00183; A61B 1/04; G02B 23/02; G02B 26/0816; G02B 23/2423

(57) ABSTRACT

A swing prism for an endoscope with a variable viewing direction, the swing prism including: a flat light inlet surface, a flat light outlet surface, and a flat mirror surface, wherein the flat light inlet surface and the flat light outlet surface are arranged at a 90° angle relative to each other and are each arranged at a 45° angle relative to the flat mirror surface, the swing prism is configured as a cylinder-section-shaped body with a base surface configured as a circle segment, and a cylindrical axis of the cylinder-section-shaped body is perpendicular to the flat light outlet surface, and in a projection onto the flat light outlet surface, the flat light inlet surface constitutes a chord which delimits the circle segment.

### CROSS-REFERENCE TO RELATED APPLICATION

The present application is a continuation of PCT/EP2015/052489 filed on Feb. 6, 2015, which is based upon and claims the benefit to DE 10 2014 202 612.1 filed on Feb. 13, 2014, the entire contents of each of which are incorporated herein by reference.

### BACKGROUND

Field

The present application relates to a swing prism for an endoscope with a variable viewing direction, having a flat light inlet surface, a flat light outlet surface and a flat mirror surface, wherein the light inlet surface and the light outlet surface are arranged at a 90° angle relative to each other and are each arranged at a 45° angle relative to the mirror surface. The present application further relates to a prism mounting arrangement with a corresponding swing prism, two stationary prisms for an endoscope with a variable viewing direction, wherein light which enters through a light inlet surface of the swing prism is deflected into an axial direction of the endoscope by means of the prisms, and a prism mounting for the swing prism as well as an endoscope with a variable viewing direction.

Prior Art

Endoscopes, such as video endoscopes, in which the light of an operative field entering at a distal tip of an endoscope shaft of the endoscope is directed through an optical system to a proximal ocular or onto one or more image sensors, are known in different designs. Thus, there are endoscopes with a direct view, of a so-called 0° viewing direction, or endoscopes with a lateral viewing direction, which have for example a lateral viewing direction of 30°, 45°, 70° or the like differing from the 0° viewing direction. The named degree numbers here mean the polar angle between the central viewing axis and the longitudinal axis of the endoscope shaft. There are also endoscopes or respectively video endoscopes with an adjustable lateral viewing direction, in which the viewing angle, i.e. the deviation from the direct view, is adjustable. Besides an adjustment of the viewing angle, i.e. of the deviation from the direct view, the viewing direction, i.e. the azimuth angle, can also be adjusted around the longitudinal axis of the endoscope shaft, in that the endoscope is rotated in its entirety around the longitudinal axis of the endoscope shaft.

European patent application EP 2 369 395 A1 shows an optical system for a video endoscope, in which a change in the viewing angle occurs in that a first prism of a prism group arranged distally in the endoscope shaft with three prisms is rotated about an axis of rotation, which lies perpendicular or respectively transverse to the longitudinal axis of the endoscope shaft. The two other prisms, which define the optical beam path together with the first prism, are not rotated.

The prism, referred to as a swing prism within the framework of the present application, is cut from a glass block. The width of the glass block is in line with the size of the beam path. The prism is received in a prism mounting and is positioned by three stop surfaces. The prism mounting itself is rotatably mounted in a mounting. In the design and production of corresponding prism groups, the alignment of the prism with respect to its axis of rotation is very important. Especially in the case of a rotation, a wobbling of the prism about the axis of rotation can lead to a considerable degradation of the image quality.

The prism mounting needed to receive the prism is very complicated and complex to produce and is thus associated with high costs. In order to produce the three stop surfaces, outlets are needed for the milling, which enlarge the required installation space for the mounting. The stop surfaces must be produced very precisely both relative to each other as well as absolutely, which is only feasible with a high investment in production and quality. The alignment of the prism at the stop surfaces is also not trivial, since the prism must be simultaneously aligned at all three surfaces without tipping.

### SUMMARY

Based on the prior art, an object is to reduce the effort to produce, assemble and align prisms and prism mountings in corresponding endoscopes with at least the same optical quality.

Such object can be solved by a swing prism for an endoscope with a variable viewing direction, having a flat light inlet surface, a flat light outlet surface and a flat mirror surface, wherein the light inlet surface and the light outlet surface are arranged at a 90° angle relative to each other and are each arranged at a 45° angle relative to the mirror surface, in which the swing prism is configured as a cylinder-section-shaped body with a base surface configured as a circle segment, and the cylindrical axis of the body is perpendicular to the light outlet surface, wherein in the projection onto the light outlet surface, the light inlet surface defines a chord which delimits the circle segment.

In the cross-section or respectively in the projection onto the light outlet surface, which is aligned perpendicular to the cylinder axis, the prism is thus no longer circular, but rather circle-section-shaped or respectively circle-segment-shaped. The light inlet surface runs like a chord through the periphery of the previously circular base surface.

The present disclosure makes use of the idea that only the light inlet surface, the light outlet surface and the mirror surface are relevant for the function of the swing prism. The side surfaces are not relevant; thus, they do not need to be configured as flat surfaces. The mounting is configured cylindrically with a slope suitable for the prism. The swing prism can thus be easily mounted on the suitable, sloping mounting surface of the prism mounting with its sloping mirror surface. Furthermore, the need for exact alignment based on three stop surfaces is no longer applicable. Thus, tipping is less probable. This construction can thus be realized very easily and cost-effectively.

The cylinder axis can be contained in the base surface, wherein the chord is distanced from the cylinder axis in a plane of symmetry of the base surface between 20% and 70% of a radius of the base surface, such as between 30% and 60%, more particularly, between 40% and 55%. Thus, more than half of the originally circular base surface is preserved. The cylinder axis is still located within the swing prism. The named areas can also guarantee a large coverage of the visual field with a simultaneously small installation space. Both the required installation space and the visual field are increasingly reduced from large to small values of the named ranges. It depends on the application and the overall optics, where the optimal compromise lies between installation space and visual field coverage, wherein average values, approximately between 30% and 60%, or between 40% and 55%, offer a very good compromise for a plurality of situations. In the case of this reduction in size, the optical axis of rotation can be removed from the cylinder axis.

The light inlet surface and the mirror surface can be cut from a cylindrical glass body. The flat light inlet surface is thus cut from the original glass cylinder in a simple manner parallel to the central cylinder axis.

The object can also be solved by a prism mounting arrangement with a previously described swing prism as well as two fixed prisms for an endoscope with variable viewing direction, wherein light which enters through a light inlet surface of the swing prism is deflected into an axial direction of the endoscope by means of the prisms, and a prism mounting for the swing prism, which can be further developed in that the prism mounting is adapted in a cylinder-section-shaped manner to the base surface of the swing prism and can be configured with an angled surface that is tilted by 45°, wherein an outer periphery of the prism mounting corresponds with an outer periphery of the swing prism.

Compared to conventional prism mounting arrangements with prism mountings that have three stop surfaces and take up considerable installation space, the prism mounting of the prism mounting arrangement can be configured to be very small. The prism mounting can also be configured in a cylinder-section-shaped manner and can be adapted to the outer periphery of the swing prism with respect to its outer periphery. The swing prism can thus be easily mounted on the suitable, sloping mounting surface of the prism mounting with its sloping mirror surface. Additionally, the outer diameters of the swing prism and prism mounting are aligned relative to each other. This can be possible in a suitable bore hole of an apparatus. Through such structure, the swing prism and the prism mounting can thus be guided in a manner precisely aligned with each other with little effort in the production and assembly of parts.

The swing prism can be arranged and fastened exclusively with its mirror surface on the angled surface of the prism mounting. This reduces the effort for the alignment and fastening and ensures a secure and spin-free mounting of the swing prism.

The inlet surface of the beam path into the swing prism must have a certain size so that the beam path is not clipped. For this, the diameter of the prism must have a certain size. The required installation space is increased by the necessary size of the swing prism. In a further development, the prism mounting can have an eccentrically arranged axis of rotation, which runs parallel to the cylinder axis through a plane of symmetry of the circle-section-shaped base surface and can be located further away from the light inlet surface than the cylinder axis. These measures reduce the required installation space. The bearing axis of the prism mounting can also be decentered towards the alignment diameter of the prism mounting. The beam path and the axis of rotation hereby remain concentric. Thus, the inlet surface of the beam path can be positioned such that more than half of the outer diameter of the swing prism remains for the alignment of the prism mounting.

In a further development, the prism mounting on the side facing away from the swing prism in extension of the axis of rotation can have an axle journal, which is rotatably mounted in a pivot bearing of a mounting of the prism mounting arrangement. This results in a single-sided mounting of the combination of the prism mounting and the swing prism, which always ensures a correct alignment of the swing prism in relation to the second prism of the prism group. Such configuration constitutes a easily realizable and cost-effective type of mounting and support.

Overall, the combination of the swing prism with the prism mounting results in considerably reduced installation space since the prism mounting no longer surrounds the swing prism. The mounting and alignment of the swing prism on one hand relative to the prism mounting and on the other hand relative to the other optical elements of the endoscope are also hereby simplified and improved.

The object is also solved by an endoscope with a variable viewing direction with the previously described prism mounting arrangement.

Further features will become apparent from the description of embodiments together with the claims and the included drawings. Embodiments disclosed herein can fulfill individual features or a combination of several features.

### BRIEF DESCRIPTION OF THE DRAWINGS

The embodiments are described below without restricting the general idea using exemplary embodiments with reference to the drawings, and for any details which are not explained further in the text express reference is made to the drawings. They show in:

FIG. 1 illustrates a schematic perspective representation of an endoscope,

FIG. 2 illustrates a schematic side view of a known prism unit,

FIG. 3 illustrates a schematic top view of a known prism unit,

FIG. 4 illustrates a schematic sectional representation through a prism mounting arrangement for the prism unit of FIG. 2,

FIGS. 5a) and 5b) illustrate a schematic perspective representation of a known prism and a prism mounting, respectively,

FIG. 6 illustrates a schematic sectional representation through a prism mounting arrangement, and

FIGS. 7a), 7b) and 7c) illustrate a schematic top view and perspective representations, respectively, of a prism mounting.

In the drawings, the same or similar elements and/or parts are provided with the same reference numbers in order to prevent the item from needing to be reintroduced.

### DETAILED DESCRIPTION

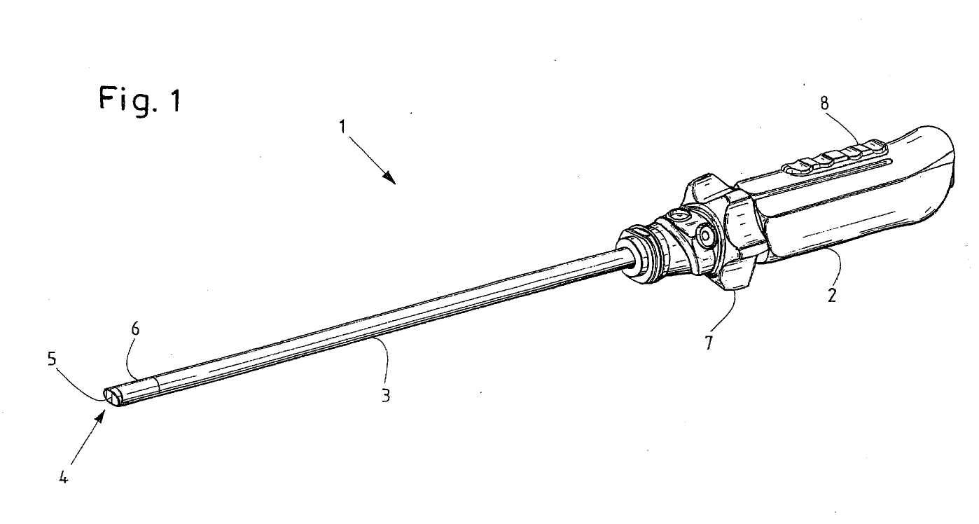

FIG. 1 shows a schematic perspective representation of an endoscope 1 with a proximal handle 2 and a rigid endoscope shaft 3. A viewing window 5 is arranged on the distal tip 4 of the endoscope shaft 3, behind which a distal section 6 of the endoscope shaft is arranged, which has a prism unit (not shown) and an image sensor unit (not shown). The endoscope 1 can be configured as a video endoscope.

The viewing window 5 on the distal tip 4 is configured to be curved and asymmetrical. The viewing window 5 is thus configured to support a variable lateral viewing angle. A change in the viewing direction, i.e. a change in the azimuth angle about the longitudinal axis of the endoscope shaft 3, is effectuated by a rotation of the handle 2 about the central axis of rotation or respectively the longitudinal axis of the endoscope shaft 3. The jacket tube of the endoscope shaft 3 is connected with the handle. The prism unit (not shown) on the distal tip 4 also rotates with the rotation of the handle 2.

The handle 2 has a first operating element configured as rotary wheel 7 and a second operating element configured as slide switch or pusher 8.

In order to maintain the horizontal position of the shown image, the rotary wheel 7 is held tight during a rotation of the handle 2. This ensures that the image sensor inside the endoscope shaft 3 does not follow the movement.

The pusher 8 is moved in order to change the viewing angle, i.e. the deviation of the viewing direction from the direct view. A pushing of the pusher 8 distally leads for example to an enlargement of the viewing angle; a retrieval of the pusher 8 proximally effectuates in this case a reduction in the viewing angle up to the direct view. The actuation of the pusher 8 involves a rotation of the image sensor in order to also maintain the horizontal position of the shown image during a twisting of the prism unit against each other.

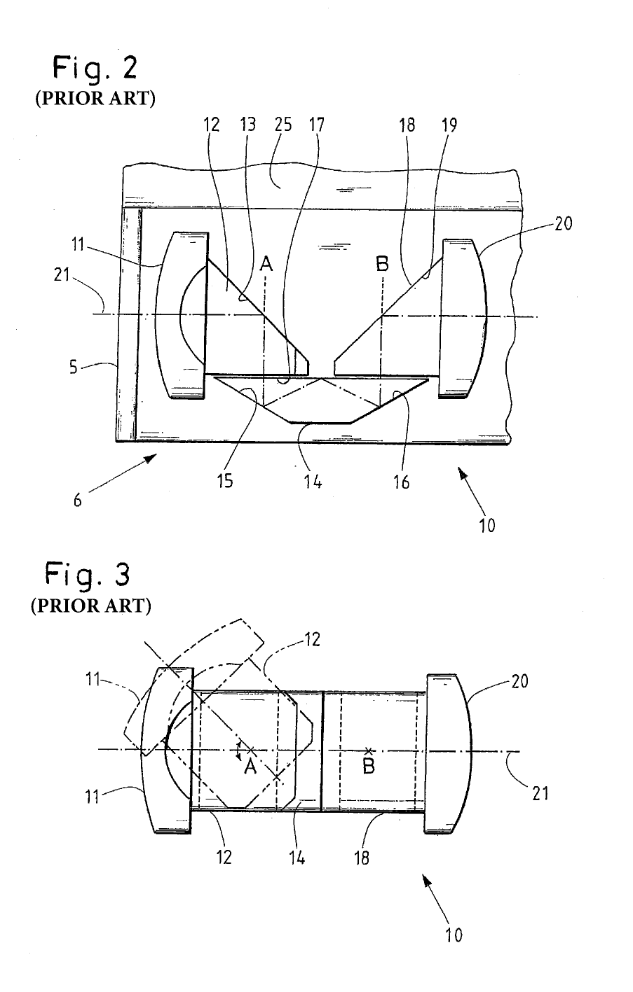

FIG. 2 shows a schematic representation of a known prism unit 10 from the side. On the left side of the picture, light from a central beam path 21, which is shown as a dashed-and-dotted line, enters through a viewing window 5 and enters into a first, distal prism 12 through an entry lens 11. The light hits the mirror surface 13 and is mirrored through total reflection or a mirroring downwards towards a second prism 14 as well as a mirror surface 15 of the second prism. The mirror surface 15 has an acute angle to the bottom side 17 of the second prism 14, so that the central beam path is first mirrored onto a central section of the bottom side 17 and from there towards a second mirror surface 16 of the second prism 14. This second mirror surface 16 also has an acute angle to the bottom side 17 so that the central beam path is in turn reflected upwards (axis B). There, the light enters a third prism 18 with a mirror surface 19, through which the light of the central beam path 21 is mirrored in turn centrally in a direction parallel to the longitudinal axis of the endoscope shaft 3 and exits the prism unit 10 through an exit lens 20. More-over, a part of an optical fiber bundle 25, by means of which light is directed from proximal to the distal tip, in order to illuminate an otherwise non-illuminated operative field, is still represented above the prism unit 10.

The first prism 12 can be turned about the perpendicular axis A in order to adjust the lateral viewing angle. The mirror surfaces 13 and 15 also thereby rotate against each other so that the horizontal position of the image, which is forwarded proximally, is changed in the case of a rotation of the first prism 12 about the axis A. This must be counterbalanced by a rotation of the image sensor(s).

FIG. 3 shows the prism unit 10 of FIG. 2 in a schematic top view. The left side shows how the first prism 12 is arranged in a 0° viewing direction (solid lines). It is also shown with dashed lines that the first prism 12 is rotated about the axis of rotation A together with the entry lens 11. In this case, the overlapping area between the mirror surfaces 13 of the first prism 12 and 15 of the second prism 14 is skewed. Accordingly, the horizontal position is also skewed.

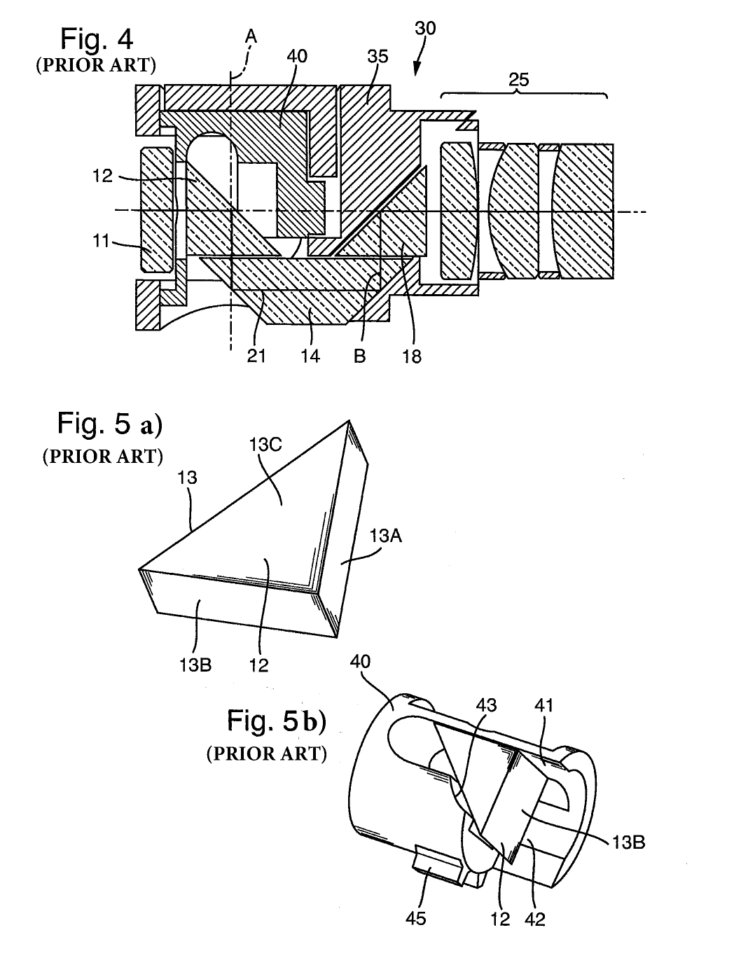

FIG. 4 shows a schematic sectional representation through a prism mounting arrangement 30, which is arranged distally in an endoscope shaft 3 of an endoscope 1, for example of FIG. 1. The beam path progresses from left to right in FIG. 4. Light first enters through an entry lens 11 and makes its way into a first swing prism 12, where it is deflected 90° downwards at a mirror surface tilted by 45° and makes its way to a second prism 14. This has two mirror surfaces that are each tilted by 45° so that the incoming light, which is represented by the central beam path 21 is deflected 90° twice and makes its way into a third prism 18, which also has a mirror surface tilted by 45° and deflects the light 90° so that it makes its way through a rear lens group 25 after the last deflection in the longitudinal direction of the endoscope. Image sensors or optical rod lens systems for the routing of the light to a proximally arranged ocular can be found behind the rear lens group 25, which are not shown in FIG. 4.

The swing prism 12 is pivotable about an axis labeled with the letter “A”, which coincides with the section of the central beam path 21 that points downwards. The swing prism 12 is mounted in a prism mounting 40, which is rotatably mounted about the axis “A” in an outer mounting 35. The second prism 14, the third prism 18 and the rear lens group 25 are also mounted in the outer mounting 35.

FIG. 5a) shows a prism that is suitable as a swing prism 12 in a schematic perspective representation, which has an isosceles, right-angled configuration, wherein the light inlet surface 13A and the light outlet surface 13B form the side and adjacent side of the right angle, while the mirror surface 13 forms the hypotenuse of the right-angled triangle, i.e. the side lying opposite the right angle. The side surfaces 13C are flat.

FIG. 5b) shows a schematic perspective representation of the swing prism 12 and of a prism mounting 40 of FIG. 4. The prism mounting 40 has a cylindrical circumferential contour and has on its inside a first stop surface 41, a second stop surface 42 and a third, sloping stop surface 43 for the swing prism 12. The three stop surfaces 41, 42, 43 are comparatively small in order to block as little as possible from the beam path. This increases the effort during the mounting and adjustment or respectively alignment of the swing prism 12 in the prism mounting 40.

Furthermore, gear wheel teeth 45 for the mechanical coupling are provided on a shifting element (not shown), which connects a sliding element on the handle with the prism mounting 40 and with which a longitudinally axial movement of the shifting element in the endoscope shaft 3 is converted into a pivoting movement of the prism mounting 40 with the swing prism 12.

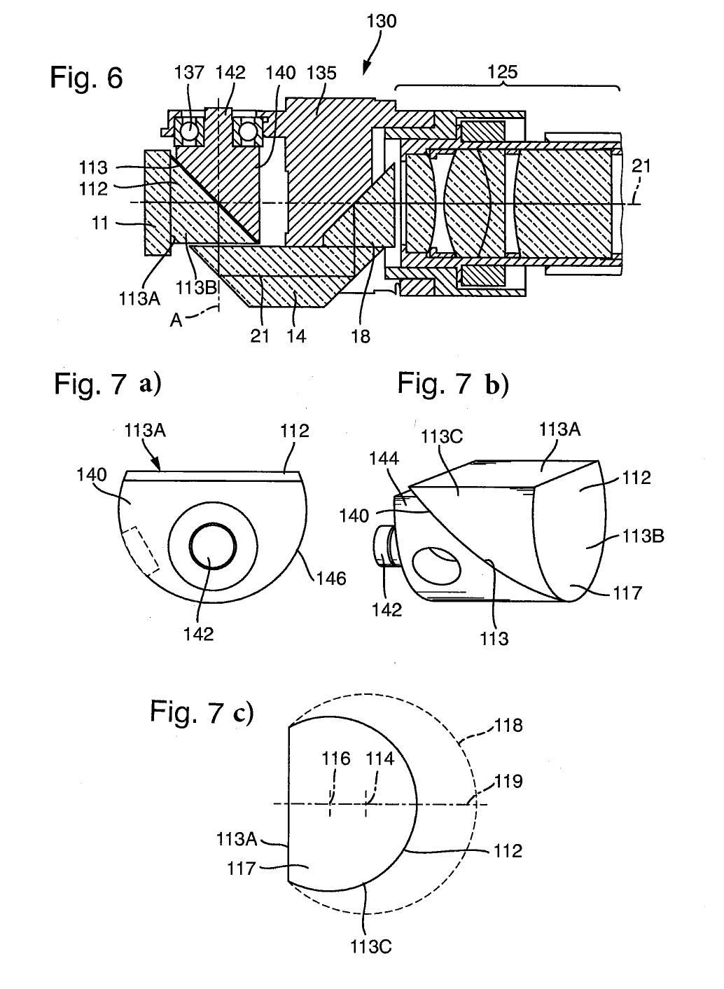

FIG. 6 shows a schematic sectional representation through a prism mounting arrangement 130 for use in the endoscope of FIG. 1. Comparable to the known embodiment of FIG. 4, according to FIG. 6 a part of the optical elements is mounted in a mounting 135 in this embodiment. This relates to a second prism 14, the third prism 18 and, except for an axial rotatability, the rear lens group 125. The mounting 135 also has a pivot bearing 137 in the distal area, in which an axle journal 142 of a prism mounting 140 is mounted. The center of the pivot bearing 137 and of the axle journal 142 coincide with the axis of rotation “A”. The prism mounting 140 has an angled surface tilted by 45°, on which a swing prism 112 is fastened with its mirror surface 113. It can be seen immediately by comparing FIGS. 6 and 4 that the installation space required for the prism mounting 140 is considerably smaller than the installation space required for the known prism mounting 40. The mounting 135 is also less projecting than the mounting 35.

FIGS. 7a), 7b) and 7c) show a schematic top view and perspective representations of a prism mounting. FIG. 7a) shows a top view of the top side of the prism mounting 140 with the axle journal 142. The outer periphery 146 of the prism mounting 140 has the shape of a circle section, i.e. a circle, which is cut through a straight chord progressing through the periphery. It can be seen in the top view that the swing prism 112 projects slightly beyond the prism mounting 140 on the side of the light inlet surface 113A. The radii of the outer diameter of the swing prism 112 and of the prism mounting 140 are matched to each other. It can also be seen that the axle journal 142 is arranged eccentrically relative to the actual center of the circular outer periphery 146 and is namely distanced further away from the straight edge.

FIG. 7b) shows a perspective schematic representation of the combination of prism mounting 140 and swing prism 112. The swing prism 112 is arranged and fastened with its mirror surface 113 on the angled mounting surface of the prism mounting 140 and protrudes slightly over the outer contour of the prism mounting 140 on the side of the light inlet surface 113A. Overall, the swing prism 112 and the prism mounting 140 form a substantially cylinder-section-shaped or respectively cylinder-segment-shaped body, i.e. a cylinder-shaped body with a cut or respectively flattened side parallel to the central cylinder axis. The light outlet surface 113B forms a cylinder end surface, which is aligned perpendicular to the cylinder axis. The cylinder end surface is located perpendicular to the light inlet surface 113A and at an angle of 45° to the mirror surface 113.

FIG. 7c) shows the effect of the decentering of the axis of rotation 114 relative to the cylinder axis 116 in the plane of symmetry 119, which progresses perpendicular through the middle of the light inlet surface 113A. In the case of the same-sized light inlet surface 113A, a swing prism, the outer contour of which is shown dashed as an alternative outer contour 118, would be necessary in the case of an axis of rotation that corresponds with the cylinder axis. The outer contour of the swing prism 112, which has a decentered axis of rotation 114, is shown with a solid line. The geometric ratios between the light inlet surface 113A and the axis of rotation 114, which determine the progression of the beam path 21 in the distal area, remained constant. However, the outer contour can turn out to be considerably smaller than in the case, in which the axial of rotation and the cylinder axis coincide. A swing prism with the alternative outer contour 118 also lies within the embodiments disclosed herein.

The degree of the reduction of the outer contour compared to the alternative outer contour 118 through parallel displacement of the axis of rotation in the axis of symmetry 119 compared to the cylinder axis is to be adjusted for the respective conditions of the endoscope 1. A considerably further decentering than that shown in FIG. 7c) would thus lead to a stronger cutting of the spatial angle, from which light can be directed through the entire system comprising the swing prism 112, the second prism 14 and the third prism 18, and namely, the area of the light inlet surface 113A, which lies close to the light outlet surface 113B.

While there has been shown and described what is considered to be preferred embodiments of the invention, it will, of course, be understood that various modifications and changes in form or detail could readily be made without departing from the spirit of the invention. It is therefore intended that the invention be not limited to the exact forms described and illustrated, but should be constructed to cover all modifications that may fall within the scope of the appended claims.

### REFERENCE LIST

1 Endoscope

2 Handle

3 Endoscope shaft

4 Distal tip

5 Viewing window

6 Distal section

7 Rotary wheel

8 Pusher

9 Jacket tube

10 Prism unit

11 Entry lens

12 Swing prism

13 Mirror surface

13A Light inlet surface

13B Light outlet surface

13C Side surface

14 Second prism

15, 16 Mirror surfaces

17 Bottom side

18 Third prism

19 Mirror surface

20 Exit lens

21 Central beam path

25 Rear lens group

30 Prism mounting arrangement

35 Mounting

40 Prism mounting

41 First stop surface

42 Second stop surface

43 Sloping stop surface

45 Gear wheel teeth

112 Swing prism

113 Minor surface

113A Light inlet surface

113B Light outlet surface

113C Outer periphery

114 Axis of rotation

116 Cylinder axis

117 Base surface

118 Alternative outer contour

119 Plane of symmetry

125 Rear lens group

130 Prism mounting arrangement

135 Mounting

137 Pivot bearing

140 Prism mounting

142 Axle journal

144 Angled surface

146 Outer periphery

What is claimed is:

1. A swing prism for an endoscope with a variable viewing direction, the swing prism comprising:
a flat light inlet surface,
a flat light outlet surface, and
a flat mirror surface,
wherein the flat light inlet surface and the flat light outlet surface are arranged at a 90° angle relative to each other and are each arranged at a 45° angle relative to the flat mirror surface,
the swing prism is configured as a cylinder-section-shaped body with a base surface configured as a circle segment, and a cylindrical axis of the cylinder-section-shaped body is perpendicular to the flat light outlet surface, and
in a projection onto the flat light outlet surface, the flat light inlet surface constitutes a chord which delimits the circle segment.

2. The swing prism according to claim 1, wherein the cylinder axis is contained in the base surface, wherein the chord is distanced from the cylinder axis in a plane of symmetry of the base surface between a range of 20% and 70% of a radius of the base surface.

3. The swing prism according to claim 2, wherein the range is between 30% and 60%.

4. The swing prism according to claim 3, wherein the range is between 40% and 55%.

5. The swing prism according to claim 1, wherein the flat light inlet surface and the flat mirror surface are cut from a cylindrical glass body.

6. A prism mounting arrangement for an endoscope with a variable viewing direction, the prism mounting arrangement comprising:
the a swing prism according to claim 1,
two fixed prisms, wherein light which enters through the flat light inlet surface of the swing prism is deflected into an axial direction of the endoscope, and
a prism mounting for the swing prism,
wherein the prism mounting is adapted to a circle-section-shaped base surface of the swing prism and the prism mounting is configured with an angled surface that is tilted by 45°, and
an outer periphery of the prism mounting corresponds with an outer periphery of the swing prism.

7. The prism mounting arrangement according to claim 4, wherein the swing prism is arranged and fastened on the angled surface of the prism mounting exclusively with its mirror surface.

8. The prism mounting arrangement according to claim 4, wherein the prism mounting has an eccentrically arranged axis of rotation which progresses through a plane of symmetry of the circle-section-shaped base surface parallel to a cylinder axis and is located further away from the flat light inlet surface than the cylinder axis.

9. The prism mounting arrangement according to claim 4, wherein the prism mounting on a side facing away from the swing prism in extension of an axis of rotation has an axle journal, which is rotatably mounted in a pivot bearing of a mounting of the prism mounting arrangement.

10. An endoscope with a variable viewing direction comprising the prism mounting arrangement according to claim 4.
